SEQUENCE LISTING (2 sequences)

SEQ ID NO: 1 - DNA, 22 bp, Artificial Sequence
  forward (upstream) primer that amplifies Human Alu Yb8
cgaggcgggtggatcatgaggt

SEQ ID NO: 2 - DNA, 20 bp, Artificial Sequence
  reverse (downstream) primer that amplifies Human Alu Yb8
tctgtcgcccaggccggact

(19) United States
(12) Patent Application Publication
(10) Pub. No.: US 2024/0307452 A1
(43) Pub. Date: Sep. 19, 2024

(54) UMBILICAL CORD BLOOD MONONUCLEAR CELLS AND RED CELL FRACTION IMPROVE NEUROGENESIS AND BEHAVIORAL RECOVERY AFTER HYPOXIA-ISCHEMIC BRAIN DAMAGE

(71) Applicant: MONONUCLEAR THERAPEUTICS LIMITED, Hong Kong (CN)

(72) Inventors: Hao LYU, Hong Kong (CN); Wai Sang POON, Hong Kong (CN); Chi Ping NG, Hong Kong (CN); Chi Chiu WANG, Hong Kong (CN); Wise YOUNG, Piscataway, NJ (US); Dongming SUN, Piscataway, NJ (US); Shong CHENG, Hong Kong (CN)

(21) Appl. No.: 18/570,823

(22) PCT Filed: Jun. 17, 2022
(86) PCT No.: PCT/IB2022/000338

§ 371 Date: Dec. 15, 2023

Related U.S. Application Data
(60) Provisional application No. 63/211,946, filed on Jun. 17, 2021.

(51) Int. Cl. A61K 35/18; A61K 35/51; A61K 39/00; A61P 25/00

(52) U.S. Cl. CPC A61K 35/18; A61K 35/51; A61K 39/461; A61K 39/46432; A61P 25/00; A61K 2239/31; A61K 2239/38

(57) ABSTRACT

The subject invention pertains to methods to treat neurological dysfunctions by replacing neurons, glia, vascular cells, or any combination thereof to repair the central nervous system and other tissues by administering umbilical cord blood (UCB), mononuclear cells (MNC), or red cell fraction (RCF) to a subject.

### CROSS-REFERENCE TO RELATED APPLICATION

This application claims the benefit of U.S. Provisional Application Ser. No. 63/211,946, filed Jun. 17, 2021, which is hereby incorporated by reference in its entirety including any tables, figures, or drawings.

### BACKGROUND OF THE INVENTION

Hypoxic-ischemic encephalopathy (HIE) is a major cause of mortality and morbidity in newborns. HIE causes cognitive loss, motor paralysis, seizures, and other neurological dysfunction, such as cerebral palsy (CP), from prolonged loss of blood flow and low oxygen during birth. Hypothermia therapy reduces neuron damage and neurological dysfunction in HIE but does not reduce mortality rate and may not improve neurological function.

Hypoxia and ischemia usually cause widespread brain damage, leading to neuronal loss in many parts of the brain, including the cortex, subcortical nuclei, and cerebellum. Strokes generally cause regional brain damage, often on one side of the brain due to clot or other emboli that occlude one or more arteries. Strokes may also result from intracerebral or subarachnoid hemorrhage, combining with hypoxia and ischemia to create greater brain damage.

Most therapeutic approaches have emphasized prevention of neuronal damage and loss. Cell death or injury occurs rapidly in a subject affected by a stroke or hypoxia-ischemia. Treatments must be instituted rapidly before the neurons become irreversibly damaged. Therefore, novel therapies that can be used to restore neurons after hypoxic-ischemia or stroke-induced neurological dysfunction are urgently needed.

Scientists and physicians have long believed people are born with the neurons that they die with. Although adult neural stem cells have been known to be present in the brain of adult animals for decades^{1,2}, neurogenesis was not seriously considered a mechanism of brain repair, outside of development³, the hippocampus⁴, and subcortical structures⁵.

Therefore, therapies that restore brain neurons and function after hypoxia and ischemia, stroke, and other brain conditions that damage and kill neurons remain a significant unmet medical need.

### BRIEF SUMMARY OF THE INVENTION

The invention disclosure provides a method to treat cell or tissue damage due to ischemia or a lack of blood flow, as caused by, for example, an occlusion of a brain blood vessel or hypoxic-ischemia resulting from blood supply and hypoxia of the brain. The treatment provides an effective amount of a composition comprising umbilical cord blood (UCB), preferably human umbilical cord blood, or two of its components: mononuclear cells (MNCs) and red cell fraction (RCF). The composition further comprise at least one pharmaceutically acceptable substance or compound for delivery to a subject, following onset of ischemia or a lack of blood flow in the brain that restores new neurons. The amount of the composition administered to the subject is one that is effective at ameliorating or mitigating at least one of the effects of ischemia or a reduced blow flow, including those resulting from the acute phase of stroke, hypoxic-ischemic encephalopathy, or their aftereffects. In certain embodiments, the present method can function, at least in part, by the ability of the composition of the subject invention to stimulate neurogenesis, gliogenesis, vasculogenesis, or any combination thereof to reduce or reverse the damage caused by ischemia or a lack of blood flow, and its aftereffects. In certain embodiments, the administered cells of the UCB, MNCs, or RCF do not cross or only a limited number cross the blood-brain barrier. The invention described herein restores the number of neurons in the brain through the creation of new neurons, a phenomenon called neurogenesis.

### BRIEF DESCRIPTION OF THE DRAWINGS

FIG. 1 is a schematic illustrating the experimental design of the study. The animals were assigned randomly to six experimental groups: Normal control (no brain injury, n=15), HIE only (n=15), HIE with sham injection (transplanted with 0.9% sodium chloride, n=15), and HIE with cell therapy (transplanted with 0.9% sodium chloride, UCB, MNCs or the RCF, n=15). Animals in the HIE with sham injection group were intravenously injected with 200 μL of 0.9% sodium chloride 24 hours after HIE modelling. In the cell therapy group, the rat pups were intravenously injected with 200 μl with number of 1×10⁷ UCB, MNCs or RCF 24 hours after HIE modelling.

FIG. 2. Negative geotaxis results on day 7 after cell transplantation (n=8). The UCB- and MNC-treated groups showed a better performance than the saline-treated HIE and HIE-only groups. “*” Indicates p<0.001, “**” indicates p<0.0001 (one-way ANOVA with Tukey's multiple comparison test).

FIG. 3. Beam balance results at 1 month after cell transplantation (n=12). The MNC-treated group showed better recovery of motor function than the HIE-only and saline-treated groups at 1 month. “*” Indicates p<0.05, “**” indicates p<0.005 (one-way ANOVA with Tukey's multiple comparison test).

FIG. 4. Beam balance results at 3 months after cell transplantation (n=12). The MNC-treated group showed better recovery of motor function than the HIE-only and saline-treated groups at 3 months. “*” Indicates p<0.05, “**” indicates p<0.005 (one-way ANOVA with Tukey's multiple comparison test).

FIG. 5 is a graph showing NeuN-positive cell counts 7 days after cell transplantation (n=8). More NeuN-positive cells were detected in cortex sections from HUCB and MNC-treated rats than in sections from HIE-only and saline-treated HIE rats. “*” Indicates p<0.05, “**” indicates p<0.005, “***” indicates p<0.0001 (one-way ANOVA with Tukey's multiple comparison test).

FIG. 6 is a graph showing NeuN-positive cell counts 1 month after cell transplantation (n=8). More NeuN-positive cells were detected in cortex sections from HUCB and MNC-treated rats than in sections from HIE-only and saline-treated HIE rats. “*” Indicates p<0.05, indicates p<0.005, “***” indicates p<0.0001 (one-way ANOVA with Tukey's multiple comparison test).

FIG. 7 is a graph showing NeuN-positive cell counts 3 months after cell transplantation (n=8). More NeuN-positive cells were detected in cortex sections from HUCB and MNC-treated rats than in sections from HIE-only and saline-treated HIE rats. “*” Indicates p<0.05, “**” indicates p<0.005, “***” indicates p<0.0001 (one-way ANOVA with Tukey's multiple comparison test).

FIGS. 8A-8B. (FIG. 8A) Standard curve and (FIG. 8B) expression of human Alu Yb8 in selected organs based on real-time PCR results (n=3 rat pups). The liver showed the highest number of injected MNCs, followed by the lungs and heart. Several cells were also detected in brain tissue.

FIG. 9 showed NeuN staining of control, UCB and MNC-treated rats' motor cortex.

FIGS. 10A-10B. Results of Morris water maze training trials at (FIG. 10A) 1 month and (FIG. 10B) 3 months after cell transplantation (n=12). On day 5 of training at 1 month, the time taken to find the escape platform was shorter in the UCB- and MNC-treated groups than in the saline-treated HIE groups. At 3 months, no difference in the water maze test performance was observed between any group, suggested the spatial learning and memory have not improved further. “*” Indicates p<0.05 (one-way ANOVA with Tukey's multiple comparison test).

### BRIEF DESCRIPTION OF THE SEQUENCES

SEQ ID NO: 1: forward (upstream) primer that amplifies Human Alu Yb8

SEQ ID NO: 2 reverse (downstream) primer that amplifies Human Alu Yb8

### DETAILED DISCLOSURE OF THE INVENTION

### Selected Definitions:

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, an “isolated” or “purified” compound is substantially free of other compounds, particularly compounds in the natural environment of the compound or the environment in which the compound in synthesized. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by cell counting method, cytometer, immunoblot, column chromatography, thin layer chromatography, or high-performance liquid chromatography (HPLC) analysis.

As used herein, the term “subject” refers to a mammal or other animal. For example, the term “subject” is intended to include organisms, such as, for example, animals, which are susceptible to or afflicted with neurological dysfunctions. Examples of subjects include mammals, such as, for example, humans, dogs, cows, horses, pigs, sheep, goats, cats, mice, rabbits, rats, and transgenic non-human animals. In certain embodiments, the subject is a human, such as, for example, a human suffering from, at risk of suffering from, or susceptible to neurological dysfunctions. The subject can be any ages, including neonate, infant, juvenile, adult, or elderly subject.

As used herein, the term “pharmaceutically acceptable” means compatible with the other ingredients of a pharmaceutical composition and not deleterious to the recipient thereof. As used herein, a “pharmaceutical” refers to a substance or compound manufactured for use as a medicinal and/or therapeutic drug.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, “treatment”, “treating”, “palliating” and “ameliorating” (and grammatical variants of these terms), as used herein, are used interchangeably. These terms refer to an approach for obtaining beneficial or desired results including, but not limited to, therapeutic benefits. A therapeutic benefit is achieved with the eradication or amelioration of one or more of the physiological symptoms associated with the underlying disease such that an improvement is observed in the subject, notwithstanding that the subject may still be afflicted with the underlying disease. A treatment includes delaying the appearance of a disease or condition, delaying the onset of symptoms of a disease or condition, slowing, halting, or reversing the progression of a disease or condition, or any combination thereof. The terms refer to eradicating, reducing, ameliorating, or reversing a sign or symptom of a health condition, disease or disorder to any extent, and include, but do not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, the phrase “executive functions” refers to a set of cognitive abilities that control and regulate other abilities and behaviors. Executive functions are high-level abilities that influence more basic abilities like attention, memory and motor skills. The executive functions are necessary for goal-directed behavior, and include the ability to initiate and stop actions, to monitor and change behavior as needed, and to plan future behavior when faced with novel tasks and situations. Executive functions allow subjects to anticipate outcomes and adapt to changing situations. The ability to form concepts and the ability to think abstractly are often considered components of executive function.

As used herein the phrase “brain dysfunction” or “neurological dysfunction” is intended to define a group of disorders in which one or more regions of a subject's brain and/or neurological system or function are dysfunctional compared to the predetermined functionality of the regions of the brain or neurological system. Examples of brain or neurological dysfunctions include hypoxic ischemic encephalopathy, cerebral palsy, epilepsy, cognitive impairment, stroke, headache, and memory loss. However, the present invention is not to be construed as being limited to the treatment of these listed examples.

### Compositions for Use in the Subject Methods

In certain embodiments, various compositions can be administered to a subject suffering from a brain dysfunction. The compositions that can be administered to a subject in need thereof include umbilical cord blood (UCB), preferably human umbilical cord blood, and its two components mononuclear cells (MNCs) and red cell fraction (RCF).

The initial separation of UCB is by centrifugation, yielding three fractions: platelet rich plasma (PRP), mononuclear cells (buffy-coat layer), and red cell fraction⁶. The percentage of red blood cells of UCB at birth is higher than peripheral blood, about 56% to about 61% red blood cells by volume^{7,8,9,10}. The red cell fraction (RCF) includes mostly red blood cells, as well as about 10% to about 20% neutrophils. The mononuclear cells (MNC) or buffy-coat layer contains lymphocytes at a concentration of about 20% to about 70% or about 25% to about 59% by volume and monocytes at a concentration of about 5% to about 15%, about 6.7% to about 10.7%, or about 8.7% by volume, as well as stem cells (CD34+ and CD133+), endothelia progenitor cells, hematopoietic progenitor cells, endothelial and other progenitor cell, immature lymphocytes at a concentration of about 1% to about 5% or about 2% to about 3% by volume, hematopoietic progenitor cells, endothelia progenitor cells, immature lymphocytes, mesenchymal stem cells, and multi-lineage differentiating stress enduring (MUSE) cells.

In certain embodiments, human proteins found in UCB include, for example, serum albumin, globulins, and fibrinogen. MNCs contain enriched lymphocytes at a concentration of about 30% to about 70%, about 38% to about 58%, or about 48% by volume, monocytes at a concentration of about 5% to about 40%, about 20% to about 30%, about 8% to about 20%, or about 14% by volume. In certain embodiments, the lymphocytes can have cell surface markers CD4, CD8, CD19, CD20, CD16, CD56, or a combination thereof; monocytes can have cell surface marker CD14; and progenitor cells can have surface antigens CD45, CD34, CD133, CD14, or a combination thereof. RCF can contain mainly red blood cells at a concentration of about 38% to about 58% by volume and neutrophils at a concentration of about 17% to about 88% or about 68% by volume of white blood cells. Each of UCB, MNCs, and RCF can be prepared using methods that are well-known in the art. In certain embodiments, the MNC and RCF can be processed by a sedimentation-based process method. MNC and RCF can be separated and collected by density gradient. In preferred embodiments, the ThermoGenesis (Rancho Cordova, CA) X-series system, a commercial cell process system, can be used to separate MNC and RCF and, also, for UCB separation.

In certain embodiments, the UCB, MNCs, or RCF can be derived from a species distinct from the subject that is being treated by the composition comprising UCB, MNCs, or RCF. For example, the UCB administered to a rat (Rattus norvegicus) can be derived from humans (Homo sapiens) or, for example, the UCB administered to a human can be derived from a rat. In certain embodiments, the UCB, MNCs, or RCF can be derived from the same species as the subject that is being treated by the composition comprising UCB, MNCs, or RCF.

An embodiment of the current invention also provides a composition comprising a UCB, MNC, or RCF and a pharmaceutically acceptable carrier and/or excipient.

The pharmaceutically acceptable carrier and/or excipient comprise substances, such as an inert vehicle, or pharmaceutical acceptable adjuvants, preservatives, etc. Examples pharmaceutically acceptable substances are well known to a person of ordinary skill in the art and such embodiments are within the purview of the current invention. The pharmaceutical composition may be a liquid formulation. When the pharmaceutical composition is a liquid formulation it may be formulated as an oral solution, a suspension, an emulsion or syrup.

Pharmaceutical compositions, as disclosed herein, can be formulated in accordance with standard pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999 Marchcel Dekker, New York) known by a person skilled in the art.

Carriers and/or excipients, according the subject invention, can include any and all solvents, diluents, buffers (such as neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol), and the like, including compounds that allow the compositions of the subject invention to have the same osmotic pressure as blood and lacrimal fluid. The use of carriers and/or excipients in the field of drugs and supplements is well known. Except for any conventional media or agent that is incompatible with the target health-promoting substance, carrier or excipient use in the subject compositions may be contemplated.

In preferred embodiments, the UCB, MNCs or RCF compositions are formulated to be administered via, for example, injection, which includes intravenous injection, intravascular injection, intraperitoneal injection, intramuscular injection, intrathecal administration, subcutaneous injection, intranasal administration, subarachnoid injection, localized injection, catheter administration, systemic injection, parenteral administration, intracranial injection, intra-arterial injection, intraplacental injection, intrauterine injection, intraventricular administration, intracisternal administration, intrastriatal administration, intranigral administration, intracerebral administration, intra-tissue, epidural, intradural, intrameningeal, intraspinal, surgical injection into a tissue of interest or via direct application to tissue surfaces (e.g., during surgery or on a wound).

In one embodiment, the adjuvant composition can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600, and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions (such as physiological saline) and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance is an acceptable isotonic solution, such as 5% dextrose or 0.9% sodium chloride; or 0.01-0.2% dipalmitoyl diphosphatidylcholine in USP WFI and 1 to 10% squalene or parenteral vegetable oil-in-water emulsions.

Further components can be added to the compositions as are determined by the skilled artisan, for example, buffers, carriers, viscosity modifiers, preservatives, flavorings, dyes, and other ingredients specific for an intended use. One skilled in this art will recognize that the above description is illustrative rather than exhaustive. Indeed, many additional formulations techniques and pharmaceutically-acceptable excipients and carrier solutions suitable for particular modes of administration are well-known to those skilled in the art.

### Methods of Treating Brain Dysfunctions

In certain embodiments, the compositions and methods of the subject invention can be used to treat an injury, dysfunction, disorder, or disease in the brain or neurological system, particularly in the cortex of the brain. In certain embodiments, the injury, dysfunction, disorder, or disease can be the injury, dysfunction, disorder, or disease associated with or resulting from oxygen deficiency and/or circulation insufficiency. In certain embodiments, the injury, dysfunction, disability or disease can be caused by hypoxia and/or ischemia. In certain embodiments, the hypoxia and/or ischemia can be local. In certain embodiments, the hypoxia and/or ischemia can be diffuse. In certain embodiments, the disease can be hypoxic ischemic encephalopathy.

In a further embodiment, the injury, dysfunction, disorder, or disease can be an injury, dysfunction, disorder, or disease associated with or resulting from an inadequate blood supply. In certain embodiments, the injury, dysfunction, disorder or disease can result from stenosis or occlusion or rupturing of an artery or vein, such as, but not limited to, thrombus or occlusion caused by an embolus. In certain embodiments, the injuries, dysfunctions, disorders or diseases can be associated with or resulting from an infarction or ischemia. In certain embodiments, the injuries, dysfunctions, disorders, or diseases can be associated with or result from necrosis. In certain embodiments, the infarction can be a cortical infarction. In certain embodiments, the injury, dysfunction, disorder, or disease can be a stroke or a hemorrhagic stroke, including intracerebral hemorrhage or subarachnoid hemorrhage.

In further embodiments, other related brain dysfunction can be treated by the subject methods, such as, for example, cerebral palsy, epilepsy, cognitive impairment, headache, and memory loss.

In certain embodiments, the quantity of cells to be administered will vary for the subject being treated. In a preferred embodiment, between about 1×10³ to about 1×10¹¹, more preferably about 1×10⁴ to about 1×10⁹, more preferably about 1×10⁵ to about 1×10⁷ and most preferably about 1×10⁷ cells from UCB, MNC, or RCF can be administered to a subject. The precise determination of what would be considered an effective dose may be based on factors individual to each patient, including their size, age, disease or injury, size of damage caused by the disease or injury and amount of time since the damage occurred.

In certain embodiments, the administration of UCB, MNC, or RCF to a subject can stimulate endogenous neural stem cells to make new neurons that replace lost neurons. In certain embodiments, the administration of UCB, MNC, or RCF to a subject can induce gliogenesis to repair the blood brain barrier. In certain embodiments, the administration of UCB, MNC, or RCF to a subject can induce vasculogenesis to repair injured blood vessels. The organs can be, for example, brain, spinal cord, bone, bone marrow, heart, lung, liver, pancreas, spleen, stomach, intestines, kidneys, muscles, guts, skin, hair, retina, hair organs, or any combination thereof. In certain embodiments, the administration of UCB, MNC, or RCF can release growth factors, cytokines, exosomes, cell free nucleic acids and other paracrine factors that can penetrate across the blood brain barrier and other vascular barriers to stimulate neural stem cells and mesenchymal stem cells to repair tissues.

In certain embodiments, the administration of UCB, MNC, or RCF can enhance locomotor function, motor coordination, sensory perception, cognition, visual and auditory function, smell and taste function, memory, or any combination thereof. In certain embodiments, the administration of UCB, MNC, or RCF can replace neurons, generate new neurons (i.e., neurogenesis), replace astrocytes, generate new astrocytes (i.e., gliogenesis), replace vascular cells, generate new vascular cells (vasculogenesis), and restore function in the motor cortex, thalamus, hypothalamus, hippocampus, sensory cortex, auditory cortex, basil ganglia, cerebellum, olfactory bulb, retina, auditory hair cells, sensory receptors, or any combination thereof.

In certain embodiments, either none or limited numbers of the administered cells and/or compositions of UCB, MNC, or RCF can cross the blood-brain barrier to a subject. In certain embodiments, the, for example, intravascularly or intra-tissue administered compositions can remain in the, for example, circulatory system or in the tissue in which the UCB, MNC, or RCF are administered. In certain embodiments, less than about 50 cells, 40 cells, 30 cells, 25 cells, 20 cells, 15 cells, 10 cells, 5 cells, or fewer cells per 25 mg of brain tissue can cross the blood-brain barrier of the subject upon administration of UCB, MNC, or RCF or a composition comprising UCB, MNC, or RCF.

The UCB, MNC, or RCF can be administered into epithelial tissue, connective tissue, or muscular tissue and can be entirely retained or nearly entirely retained in the epithelial tissue, connective tissue, or muscular tissue, i.e., the UCB, MNC, or RCF either do not contact nervous tissues or a limited amount of the UCB, MNC, or RCF contact the nervous tissue. In an alternative embodiment, the UCB, MNC, or RCF can be administered into epithelial tissue, connective tissue, brain/nervous tissue, or muscular tissue.

In certain embodiments, production of new neurons are induced by administration of UCB, MNC, or RCF, and the new neurons are derived from the neural stem cells that are already in the brain of the subject before administration of UCB, MNC, or RCF. In certain embodiments, cytokines, growth factors, exosomes, cell free nucleic acids, proteins, or other components found in the infused UCB, MNC, or RCF, including, for example, by intravenous, intracerebral, subarachnoid, intra-arterially, or intranasal routes, can stimulate neurogenesis, gliogenesis, and/or mesenchymal genesis in the brain. In certain embodiments, administration of RCF, which lacks stem cells or progenitor cells, induces neurogenesis, gliogenesis, and mesenchymal genesis. In certain embodiments, exosomes can be isolated from, for example, UCB, MNC, RCF, or any combination thereof. The isolated exosomes can be administered to the subject. In certain embodiments, the new neurons produced after the administration of UCB, MNC, or RCF can express Ki67 and Nestin, Ki67 and DCX, Ki67 and PSA-NCAM, or Ki67 and NeuN. In certain embodiments, the expression of Ki67 and Nestin, Ki67 and DCX, Ki67 and PSA-NCAM, or Ki67 and NeuN can occur in less than about 9 months, about 8 months, about 7 months, 6 months, about 5 months, about 4 months, about 3 months, about 2 months, about 1 month, about 3 weeks, about 2 weeks, about 1 week, about 6 days, about 5 days, about 4 days, about 3 days, about 2 days, or about 1 day, or about 1 day to about 9 months, about 1 week to about 8 months, about 2 weeks to about 7 months, about 3 weeks to about 6 months, about 1 month to about 6 months, or about 3 months to about 6 months.

In certain embodiments, the compositions of the subject invention can ameliorate at least one symptom or treat at least one neurological dysfunction within an acute timeframe, a subacute timeframe, or a chronic timeframe. An acute time frame can be about 10 minutes to about 24 hours, about 30 minutes to about 12 hours, or about 1 hour to about 4 hours after administration. A subacute time frame can be about 24 hours to about 10 days, about 48 hours to about 7 days, or about 2 days to about 3 days after administration. A chronic time frame can be about 1 week to about 1 year, about 2 weeks to about 6 months, about 4 weeks to about 3 months, or about 1 month to about 3 months.

### Monitoring of Subjects after Administration of UCB, MNC, or RCF

Following administration of UCB, MNC, or RCF, the therapeutic effect of the UCB, MNC, or RCF may be monitored. For example, the functionality of UCB, MNC, or RCF administration to treat a brain dysfunction may be monitored by analyzing behavioral studies before and after administration of the UCB, MNC, or RCF. The behavioral studies can be, for example, negative geotaxis and beam balance.

In certain embodiments, the therapeutic effects can be monitored by Hammersmith Infant Neurological Examination, Hammersmith Neonatal Neurological Examination, Griffiths Mental Development Scale, Child Behavior Checklist for Attention Deficit, Quantitative Checklist for Autism in Toddlers, hemoglobin level, oxygenation levels, oxidative stress levels (change in the baseline isoprostane level), hematocrit levels, mortality and/or adverse outcome events, or the frequency of requirements of packed cell transfusion.

### MATERIALS AND METHODS

### Human Umbilical Cord Blood, Mononuclear Cell, and Red Cell Fraction Preparation

The collection of Umbilical Cord Blood (UCB) was approved by the Joint Chinese University of Hong Kong-New Territories East Cluster Clinical Research Ethics Committee (CREC Reference Number 2016.250). Blood was collected from the umbilical cord and connected placenta from consented mothers in the Department of Obstetrics and Gynecology, The Chinese University of Hong Kong at Prince of Wales Hospital. The mother has provided signed consent for the use of these materials. The blood was collected in bags containing anticoagulant. HUCB, MNC, and the RCF were prepared by density-gradient centrifugation based on laboratory protocols and will be stored in liquid nitrogen if no immediate clinical or research requests were made. The cells were re-suspended in 0.9% sodium chloride, and their cell numbers were determined. The cell viabilities were confirmed to be higher than 90%. The qualified collections of human UCB were processed by our collaborator Mononuclear Therapeutics Ltd (MT, Hong Kong SAR, China) using the latest FDA approved SynGenX-1000 system and CryoPRO-5 Processing and Storage Bag (SynGen Inc, San Carlo, CA, United States).

### HIE Animal Model:

All animal experimentation protocols followed the guidelines for the care and use of animals approved by the Clinical Ethics Committee and Animal Ethics Committee of the Chinese University of Hong Kong (2016.250 and 16-208-MIS-5-C, respectively). Modified bilateral HIE model was used to induce hypoxic-ischemic brain damage in 7-day-old neonatal rats. All animals were housed under a 12-h light/12-h dark cycle with free access to food and water. The rat pups were fully anesthetized with isoflurane (3-4% for induction and 1-2% for maintenance), and a small incision was made in the middle of the neck using a #11 scalpel. Using blunt dissection, the connective tissue and muscles were separated to clearly visualize blood vessels under a microscope. To establish the bilateral HIE model, both common carotid arteries (CCAs) were ligated with 6-0 silk suture to a plastic tube placed next to the arteries and tied together at the node. After the incision was closed with a suture, the rat pups were placed in a hypoxia chamber containing 8% oxygen balanced with 92% nitrogen for 60 minutes. A heating pad was used to maintain the pups' body temperature in the chamber. At the end of hypoxia exposure, the rat pups were removed from the chamber and anesthetized with isoflurane. Their incisions were opened, and the nodes on both CCAs were released. The incisions were then closed with 6-0 suture, and the pups were allowed to recover in a recovery box. Buprenorphine (Buprenex) (0.05 mg/kg) was administered to each rat pup for 6 hours for postoperative analgesia.

### Experimental Grouping:

The rat pups were assigned randomly to six experimental groups: Normal control (no brain injury), HIE only, HIE with sham injection (transplanted with 0.9% sodium chloride), and HIE with cell therapy (UCB, MNCs or the RCF).

The rat pups in the HIE with sham injection group (n=15) were intravenously injected with 200 μL of 0.9% sodium chloride 24 hours after HIE modelling (i.e., bilateral CCA occlusion followed by hypoxia). In the cell therapy group (n=15), each rat pup was intravenously injected with 1×10⁷ (200 μl volume) of UCB, MNCs or RCF 24 hours after HIE modelling.

### Cell Transplantation:

For cell transplantation, the rat pups in the cell therapy group were fully anesthetized with isoflurane (3-4% for induction and 1-2% for maintenance), and a small incision was made in the left side of the neck using a #11 scalpel. Using blunt dissection, the connective tissue and muscles were separated to visualize the left external jugular vein under a microscope. Using a 30 G insulin syringe, 1×10⁷ cells in 200 μl were slowly injected in the direction of the heart over a period of 10 minutes. Subsequently, the syringe was withdrawn, pressure was applied to the wound to stop bleeding and the incision was closed with a suture.

### Real-Time PCR

Human Alu Yb8 was detected 2 days after cell transplantation to determine the migration of HUCB and MNC in the pups' brains. The rat pups were anesthetized with ketamine (75 mg/kg) and xylazine (5 mg/kg) and subjected to transcardiac perfusion with 0.9% sodium chloride followed by RNA later (Invitrogen™ RNAlater™ Stabilization Solution, AM7021). Total DNA was extracted from the brain, heart, liver, lungs, stomach, spleen and kidneys using a DNA extraction kit (Qiagen 69506) according to the manufacturer's instructions. The standard curve was prepared. The primer set for the human Alu Yb8 sequence (sense 5′-CGAGGCGGGTGGATCATGAGGT-3′ (SEQ ID NO: 1); antisense 5′-TCTGTCGCCCAGGCCGGACT-3′ (SEQ ID NO: 2)) was provided by our collaborator at Rutgers University (United States). Realtime PCR was performed with 300 ng of DNA and PowerUp SYBR Green Master Mix (Thermo Fisher, New York, NY, United States, A25741) using the QuantStudio™ 12K Flex System under the following conditions: 50° C. for 2 min and 95° C. for 10 min, followed by 40 cycles of 95° C. for 15 s, 62° C. for 15 s and 72° C. for 30 s. The PCR data were analyzed using the Thermo Fisher online system (see worldwide website: apps.thermofisher.com/apps/spa/#/dashboard).

### Behavioral Assessments:

### Short-term Assessments: Negative Geotaxis

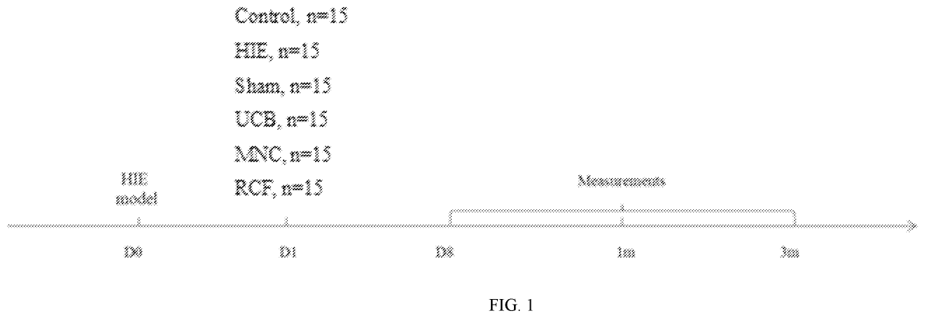

To investigate the short-term effects of cell therapy on HIE, the negative geotaxis of the rat pups in all groups was tested on day 7 after cell transplantation (FIG. 1). Briefly, the rat pups were placed on an inclined board (40°) with their head in the downward position. The time taken by the rat pups to turn 180° was recorded up to a maximum cut-off time of 60 seconds; i.e., the time to rotate for the rat pups that failed to rotate was recorded as 60 seconds.

### Long-term Assessments:

### Beam Balance

To investigate the long-term effects of cell therapy, the rat pups in all groups were subjected to the beam balance test to assess motor function assessment at 1 month and 3 months after cell transplantation (FIG. 1).

For the beam balance test, a 100-cm-long, 1.75-cm-wide beam was set 90 cm above the floor. The test was conducted in an empty cage, and bedding was placed under the beam to protect the pups from injury if they fell. On the testing day, the rat pups were placed on the beam for 1 minute, and the duration on the beam and balance score of each pup were recorded. Scoring was determined according to the following scale: 1, a stable balance posture; 2, a shaky posture indicated by grasping of the beam; 3, a balancing attempt by hanging on the beam; 4. falling off the beam after 10 seconds; 5, balancing attempt by hanging on the beam, followed by falling off the beam in 10 seconds; and 6, falling off the beam without hanging on. Each rat pup was tested three times, with a resting interval of 5 minutes between each attempt.

### Morris Water Maze

As the hippocampus was affected first after HIE, we assessed spatial-learning memory which correlated to hippocampus function. For this test, a 200-cm-diameter maze and a 10-cmdiameter escape platform were used, with four visual direction symbols placed as cues on the four tank walls. The water was maintained at room temperature. A camera connected to a computer was secured on the tank to record the pups' swimming routes. The water tank was divided into four quadrants labeled as northeast (NE), southeast (SE), southwest (SW), and northwest (NW), and the escape platform was placed in the NW quadrant. Each rat pup was tested in four trials; the pup was placed facing the tank wall in a different quadrant in each trial, starting clockwise from NE to NW. The time taken by the pup to find the escape island in each trial (from each starting quadrant) was recorded. The cut-off time to reach the island was set at 120 s, and a 15-s interval was allowed between each trial (Vorhees and Williams, 2006). For this trial, the escape island was removed, and the animal was placed facing the tank wall at a new start position, viz. in the quadrant (SE quadrant) opposite to that in which the island was originally located (NW quadrant). The cut-off time to complete the trial was set at 60 s. A new start position in the probe trial ensures that the testing animal's spatial preference is a reflection of the memory of the goal location, rather than a preference for a specific swimming path. The cumulative distance (distance between the pup and the center of the island at every 200 ms) and island crossing (number of times the pup entered the island location) were recorded to evaluate the pups' reference memory.

### Immunohistochemistry

After behavioral assessments on day 7 and at 1 month and 3 months after cell transplantation, the rat pups were anesthetized by ketamine (75 mg/kg) and xylazine (5 mg/kg) and then subjected to transcardiac perfusion with 0.9% sodium chloride followed by 4% paraformaldehyde (PFA). The brains were removed, stored in 4% PFA for 24 hours, and then dehydrated in an alcohol gradient (50%, 70%, 90% and 100%). The dehydrated brains were embedded in paraffin, and 5-μm-thick sections were cut.

For the immunohistochemistry analysis, paraffin-embedded sections were dewaxed in xylene three times (5 minutes each time), followed by rehydration in an alcohol gradient (100%, 95% and 70%; two times per concentration). The sections were washed with phosphate-buffered saline (PBS) two times (5 minutes each time). Antigen retrieval (pre-treatment for staining) was performed by heating the sections in antigen retrieval buffer (10 mM sodium citrate, 0.05% Tween 20, pH 6.0) in a microwave for 20 minutes. After cooling them to room temperature, the sections were washed with PBS two times and incubated in blocking buffer (5% normal goat serum and 0.1% Triton X-100) for 1 hour, followed by incubation with an anti-NeuN primary antibody (Millipore (Burlington, MA), MAB377, Mouse, 1:200 dilution) at 4° C. overnight. NeuN is a neuronal nuclear maker protein. The sections were washed with PBS two times and incubated with a secondary antibody (Abcam (Cambridge, United Kingdom), ab97023, Goat Anti-Mouse IgG, HRP, 1:500) for 2 hours at room temperature. Subsequently, the sections were washed with PBS two times and then stained with 3,3′- diaminobenzidine for immunohistochemical analysis. Positive cell signals were recorded using an imaging microscope under 100x magnification (Nikon Fi-3, Tokyo, Japan). For the quantitative analysis of NeuN-positive cells, six fields were selected (three from each cerebral hemisphere of each rat pup) and NeuN-positive cells were counted using Nikon NIS Element BR software.

Histological studies also include the human cell nuclei (STEM 101), and cytoplasm marker (STEM 121), TUNEL to study for apoptosis, and Ki67 to study for cell proliferation.

### Statistical Analysis:

GraphPad Prism 8.3.0 software was used for statistical analysis and graphics production. A one-way ANOVA with Tukey's multiple comparison test was performed to assess the results of the behavioral tests and immunohistochemistry analysis. A p value of <0.05 was considered significant.

### Multiplexed Staining

Instead of the traditional immunohistochemical staining, we employed multiplex tissue fluorescent immunohistochemical staining method using Mantra™ quantitative pathology workstation (PerkinElmer, Inc., Waltham, MA, USA). This system enables multispectral staining on the same specimen regardless of antibody species. The tissue processing and detailed staining were carried out using Opal 7-Color Manual IHC KiT (Akoya Biosciences, Marlborough, MA) according to manufacturer manual as described¹¹. To obtain the optimal results, we underwent stringent testing on each primary antibody for Iba1, CD68, Nestin, DCX, PSA-NCAM, A2B5, GFAP, NeuN and Ki67 to ensure they are compatible with paraffin-embedded sections and the multispectral staining.

### Cell Counting

Prior to performing the image-processing, a spectral library was established with the Nuance Image Analysis software (PerkinElmer) using multispectral images obtained from single stained slides for each marker and matched-fluorophore to capture all the lights emitted by spectral peaks of all fluorophores. The number of total, Ki67+ and Ki67-CD68, Iba1, NeuN and GFAP cells, and Ki67+ Nestin, DCX, PSA-NCAM and A2B5 cells were counted automatically using inForm® Tissue Finder Software 14.0. For cell counting, an average of at least 6 fields for motor cortex (MC) and 2 fields for subventricular zone (SVZ) regions (with at least 500 to 1500 brain cells per slide) were captured using the Mantra® Workstation. All brain cells were counted, excluding the cells of the blood vessels. The final cell counts were averaged and compared.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

### Example 1—Brain Functions Restored in HIE Rats After Treatment with MNC, UCB, or RCF

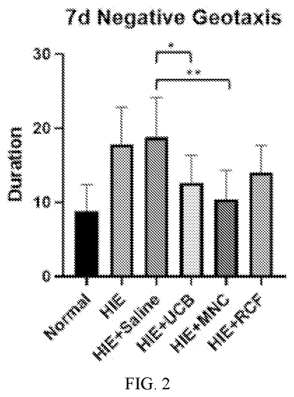

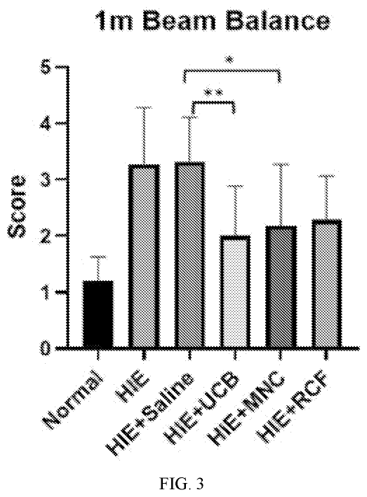

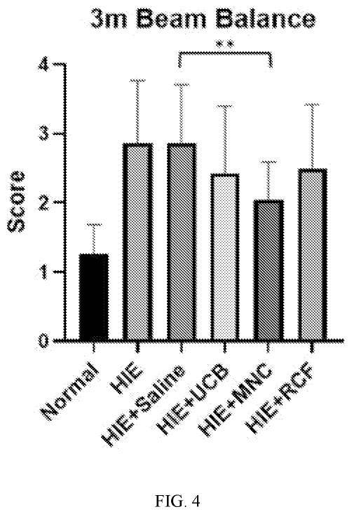

For behavioral aspect, a statistical difference in negative geotaxis appeared on Day 7 between HIE rats and MNC treated HIE rats; there is also a statistical difference between saline treated HIE rats and UCB or MNC treated rats (FIG. 2). One month after cell injection, statistical difference in Beam-Balance is demonstrated between HIE rats and UCB, MNC, or RCF treated HIE rats; there is also statistical difference between saline treated HIE rats and UCB or MNC treated HIE rats (FIG. 3). Three month after cell injection, significant difference in the Beam-Balance occurred between HIE rats and MNC treated HIE rats; there is also statistical difference between saline treated HIE rats and MNC treated HIE rats (FIG. 4). These results demonstrate improvements of locomotor function, motor coordination, sensory perception, cognition, visual and auditory function, and any combination thereof after administration of UCB, MNC, or RCF to the subject.

The treatment of neonatal rats subjected an hour of bilateral carotid occlusion and 8% hypoxia with UCB, MNC, or RCF restored the neural behavior in the rats.

### Example 2—Neurogenesis, Reconnection, and Regeration of Cortex Cells in HIE Rats After Treatment with MNC, UCB, OR RCF

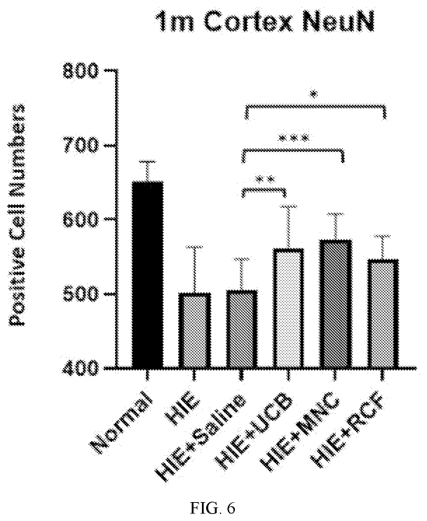

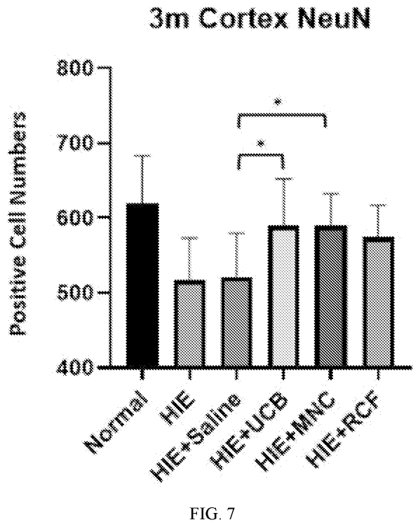

For histological analysis, 1 month after cell injection, a statistical difference in NeuN-positive cell numbers in cortex is demonstrated between HIE rats and UCB or MNC treated HIE rats; there is also a statistical difference between saline treated HIE rats and UCB, MNC, or RCF treated HIE rats (FIG. 6). Three months after cell injection, a significant difference is demonstrated between HIE rats and UCB and MNC treated HIE rats; there is also a significant difference between saline treated HIE rats and MNC treated HIE rats (FIG. 7). These results demonstrate neurogenesis, reconnection, and regeneration after administration of UCB, MNC, or RCF to the subject.

Ki67 is a cell proliferation marker for detecting cell proliferation. MNC treated neonatal cortex showed large amount of Ki67 stained cell at 1 and 3 months following cell injection, which demonstrates cell proliferation in the brain.

The treatment of neonatal rats subjected an hour of bilateral carotid occlusion and 8% hypoxia with UCB, MNC, or RCF restored the number of neurons in the brains of neonatal rats. Treatment with the MNC or RCF types clearly replaces and restores neurons damaged by severe brain ischemia and hypoxia. The fact that intravenous infusion of these MNC or RCF can reverse neuronal loss, increase gliogenesis, and increase vasculogenesis in neonatal rat brains demonstrates that UCB treatments have effects and mechanisms that are not achievable by other therapies. Cells die or are injured in a subject afflicted with a stroke or HIE. The administration of UCB, MNC, or RCF to the subject can: restore blood flow to damaged regions in a subject by increasing vasculogenesis; and stimulate the generation neurons and glial cells to restore neurological functional in a subject.

### Example 3—Quantity of MNC Cells in Organs of a Subject After Treatment with Compositions Comprising MNC

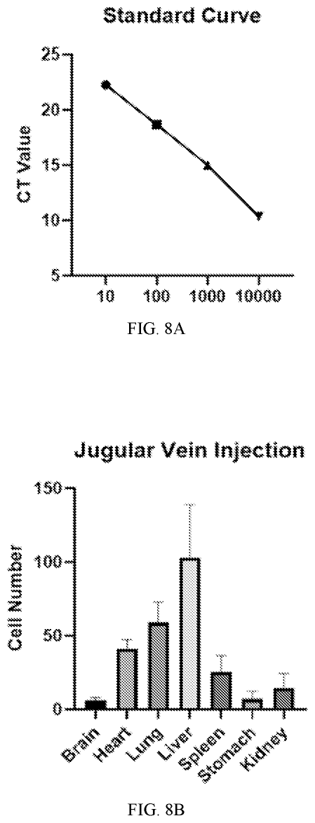

48 hours after MNC injection into the jugular vein of a subject, 25 mg of tissue from brain, heart, lung, liver, stomach, and kidney as well as 10 mg of tissue from spleen was removed. The total DNA of the tissues were extracted and purified. Human Alu Yb8 (Primer: F 5′-CGAGGCGGGTGGATCATGAGGT-3′ (SEQ ID NO: 1); R 5′-TCTGTCGCCCAGGCCGGACT-3′ (SEQ ID NO: 2)) which is commonly used human specific marker was detected by real-time PCR in 300 ng of total DNA. Then the number of cells from the injected compositions was determined (FIG. 8B). Less than about 10 cells per 25 mg of brain tissue were present in a subject after treatment with MNC, demonstrating that limited numbers of cells from compositions comprising MNC cross the blood-brain barrier.

### Example 4—Animal, Sex, Number, Mortality and Grouping

The neonatal rats were un-sexed chosen, of which 42% were female. The mortality of HIE was 30% which is consist with our previous study¹⁵, and similar with the human situation¹⁶. A total of 230 animals survived the HIE. Each neonatal rat was assigned randomly to six experimental groups: Normal control (n=12 per group), HIE only (n=12 per group), HIE with sham injection (transplanted with 0.9% sodium chloride, n=12 per group), and HIE with cell therapy (HUCB, MNC or the RCF, n=12 per group). The mortality of transplantation was 10%. Animals with too much bleeding during HIE surgery, intravenous injection, as well as blood clot found on section slides were excluded.

### Example 5—Expression of Human ALU YB8

Human UCB-derived MNC (1×10⁷) were injected intravenously into the rat pups 24 h after HIE modeling. The Alu Yb8 marker specific to these cells was detected in several organs, including the brains of the pups (n=3). A standard curve was generated to estimate the number of cells based on the cycles that reach to plateau phase (FIG. 8A). The results showed that human Alu Yb8 was detectable in all of the selected organs 2 days after intravenous cell injection, indicating that the transplanted human cells penetrated the blood-brain barrier and entered the damaged brain. Furthermore, the cell number estimates for various organs showed that the liver harvested the highest number of MNCs, followed by the lungs and heart, spleen, kidneys, stomach and brain (FIGS. 8A-8B).

### Example 6—Negative Geotaxis

In the negative geotaxis test performed on day 7 after cell transplantation (FIG. 2), rat pups in the MNC-transplanted group showed a better performance than those in the HIE-only group (p=0.0048), but their performance did not reach the level shown by the pups in the normal group. The performances of both the whole blood HUCB-transplanted (p=0.0088) and MNCs-transplanted (p=0.0001) groups were better than that of the saline-treated HIE group. However, no statistical differences in performance were found between the RCF-treated group and the saline-treated HIE (p=0.3981) and HIE-only (p=0.7046) groups.

### Example 7—Motor Behavior Test

The beam balance test was performed to assess motor behavior at 1 month (FIGS. 3) and 3 months (FIG. 4) after cell transplantation. At 1 month, the UCB-treated (p=0.0012), MNC-treated (p=0.0119), and RCF-treated (p=0.0288) groups all scored lower (indicating lesser time) than the HIE-only group, indicating that the three treated groups showed better motor balance and coordination; however, their performance did not reach the level shown by the rat pups in the normal group. Compared with the saline-treated HIE group, both the HUCB-treated (p=0.0037) and MNCs-treated (p=0.0245) groups (but not the RCF-treated group, p=0.0514) performed better, but their performance did not reach the level shown by the normal group. At 3 months, only the MNC-treated group showed a better motor performance than the HIE-only (0.0022) and saline-treated HIE (0.002) groups, but the performance did not reach the level shown by the normal group.

### Example 8—Morris Water Maze

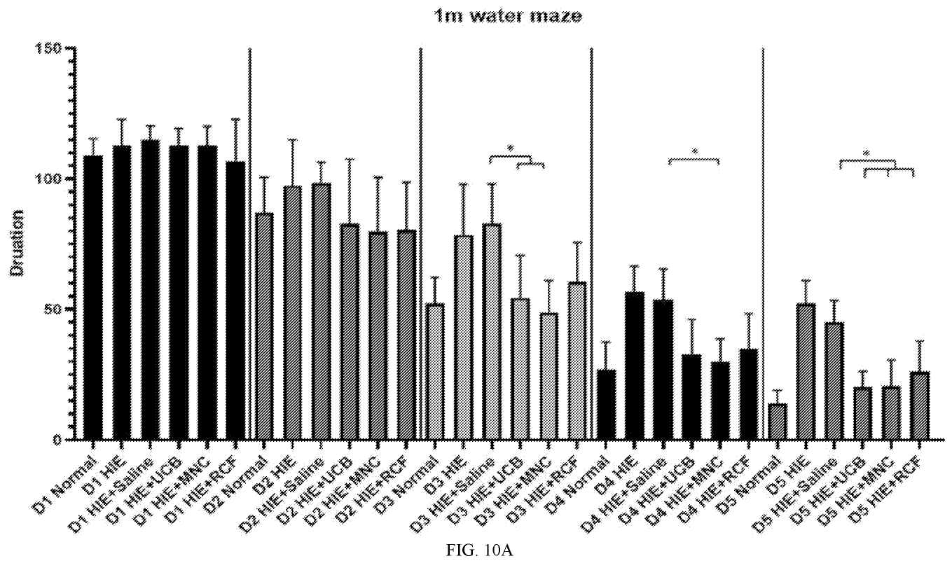

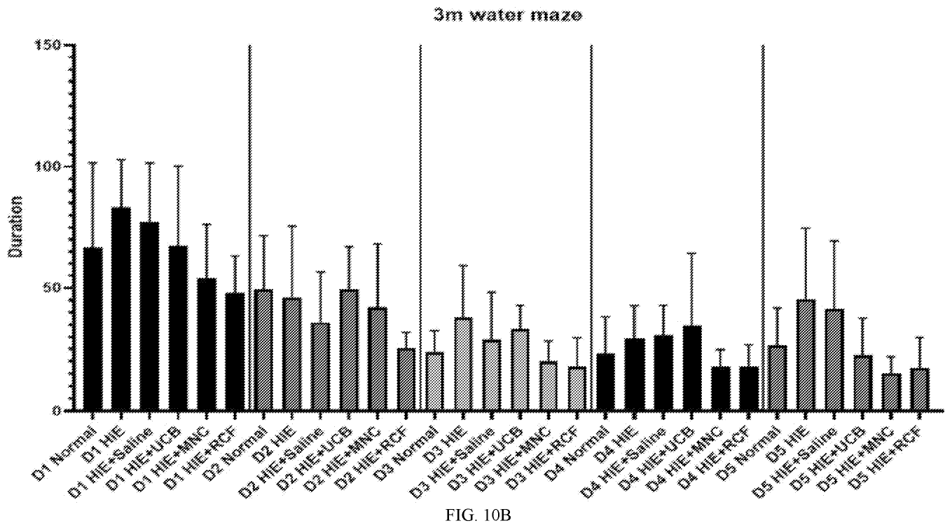

The Morris water maze test (n=12) was performed at 1 month (FIG. 10A) and 3 months (FIG. 10B) after cell transplantation to assess the spatial learning memory function of the rat pups. Five-day training trials were conducted at both time points. At 1 month, the performances of the HUCB-treated, MNCs-treated, HIE-only and saline-treated groups on day 3 were significantly different from one another, whereas on day 5, the rats in both the UCB- and MNC-treated groups found the escape platform sooner than those in the HIE-only and saline-treated groups. At 3 months, no differences were observed in the escape latency between the normal and the three treated groups. In the probe trials at 1 and 3 months, wherein the escape platform was removed, no differences were found between the groups in terms of the cumulative distance and island crossing.

### Example 9—Immunohistochemical Analysis of NeuN-Positive Cells

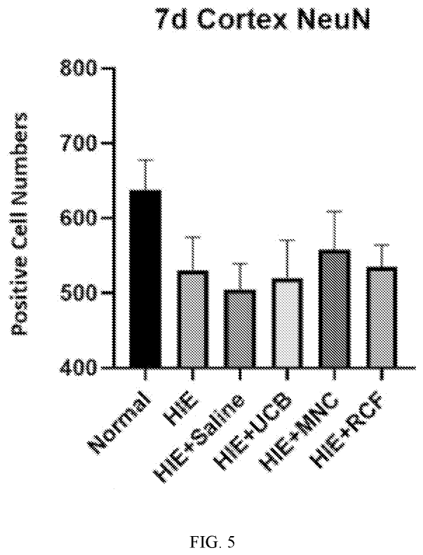

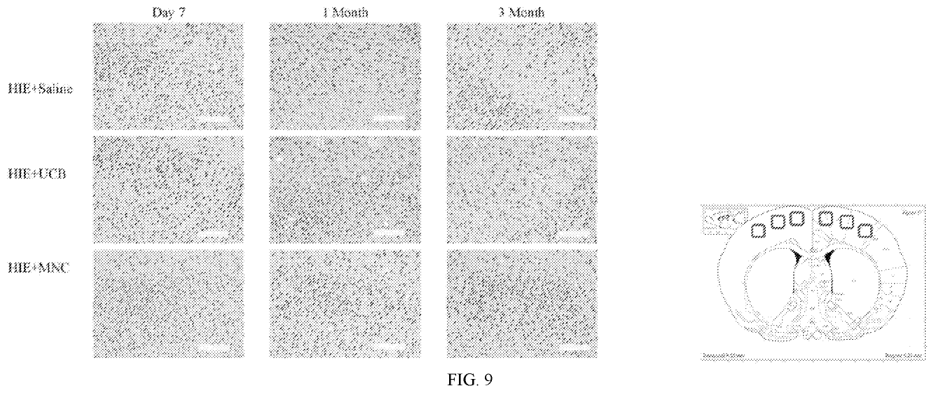

At 7 days, 1 and 3 months (FIG. 9) after cell transplantation, paraffin-embedded brain tissue sections were used for an immunohistochemistry study of neurons in the motor-sensory cortex. NeuN-positive cells were counted using software provided by Nikon. The results indicated that on day 7, all HIE groups (both with or without treatments) showed a reduced number of neurons compared with those in normal rats. Although the MNCs-treated group showed a greater number of NeuN-positive cells than the other HIE groups, the differences were not statistically significant (MNCs-treated group vs. HIE-only group, p=0.818; vs. saline-treated HIE group, p=0.1746). At 1 (FIGS. 5-7) month after cell transplantation, the UCB-treated (p=0.0024), MNC-treated (p<0.0001) and RCF-treated (p=0.0191) groups all showed bigger numbers of neurons compared with the saline-treated HIE group. Compared with the HIE-only group, the HUCB-treated (p=0.0097) and MNCs-treated (p<0.0001) groups had more neurons in the motor-sensory cortex. However, no statistical difference was observed in the number of neurons between the HIE-only and RCF-treated groups (p=0.0566). At 3 months (FIGS. 5-7), both the UCB-treated (p=0.0154) and MNC-treated (p=0.019) groups showed more neurons compared with the saline-treated HIE group. Compared with the HIE-only group, the HUCB-treated (p=0.0123) and MNCs-treated (p=0.015) groups also showed more neurons in the motor-sensory cortex. Even at this time point, however, the number of neurons between the HIE-only and RCF-treated groups (p=0.0759) and between the saline-treated HIE and RCF-treated groups (p=0.0975) were not statistically different. In line with the results of the motor behavior tests, the cell-treated groups had more neurons in the motor-sensory cortex at both 1-and 3-month time points after cell transplantation, but these numbers did not reach those found in the normal group rats.

### Example 10—Pathology Results

Further immunohistochemistry studies were performed on MC and SVZ sections of the treated animals 7-days and 1-month following the cell transplantation for Ki67^{×}positive cells, in which Ki67 marker is a nuclear protein that is strictly associated with cell proliferation¹³. The analysis studies the proliferation of various brain cells with following markers:
A2B5: A2B5, is a neuron cell surface antigen. It is a cell surface ganglioside epitope expressed in developing thymic epithelia cells, oligodendrocyte progenitors and neuroendocrine cells. A2B5 is the common target to stain for type II astrocyte, cells involved in gliogenesis and cell committed to the oligodendrocyte lineage
PSA-NCAM: PSA-NCAM (polysialylated neuronal cell adhesion molecule) is a marker of developing and migrating neurons and of synaptogenesis in the immature vertebrate nervous system.
DCX: Doublecortin (DCX)¹⁴ is a neuronal microtubule-associated protein expressed in migrating neurons of the central and peripheral nervous system during embryonic and postnatal development. In addition, the DCS is also crucial for cellular proliferation during neurogenesis.
Nestin: Nestin is intermediate filament protein expressed during embryonic development and considered largely restricted to areas of regeneration in the adult. Nestin is thought to be expressed exclusively by neural progenitor cells in the normal brain.

Regarding the HIE control group, there are no differences in macrophage and microglia in 1 month in either MC or SVZ, a decrease in neurogenesis in MC and SVZ in 7 days more so than in 1 month, and a decrease in gliogenesis in SVZ in 7 days but an increase gliogenesis in MC and SVZ in 1 month.

Regarding UCB treatment groups, there is a decrease in macrophages in SVZ in 1 month, an increase in neurogenesis in MC only in 7 days, no effects on neurogenesis in 1 month, and an increase in gliogenesis in SVZ in 1 month.

Regarding MNC treatment groups, there is no effect on macrophage and microglia in MC and SVZ in 1 month, an increase neurogenesis in MC only in 7 days, an increase in neurogenesis in MC and SVZ in 1 month, and a decrease in gliogenesis in MC in 7 days.

Regarding RCF treatment groups, there is a decrease in microglia in MC in 1 month, an increase in neurogenesis in MC only in 7 days, an increase in neurogenesis in MC only in 1 month, and a decrease in gliogenesis in SVZ in 1 month.

TABLE 1
Summary of the results.

| | | Motor Cortex (MC) | | | | Subventricular Zone (SVZ) | | | |
|---|---|---|---|---|---|---|---|---|---|
| | | HIE | UCB | MNC | RCF | HIE | UCB | MNC | RCF |
| | | vs | vs | vs | vs | vs | vs | vs | vs |
| | Markers | Con | HIE | HIE | HIE | Con | HIE | HIE | HIE |
| 7 days | Total CD68 | — | — | — | — | — | — | — | — |
| | Ki67⁻ CD68 | — | — | — | — | — | — | — | — |
| | Ki67⁺ CD68 | — | — | — | — | — | — | — | — |
| | Total Iba1 | — | — | — | — | — | — | — | — |
| | Ki67⁻ Iba1 | — | — | — | — | — | — | — | — |
| | Ki67⁺ Iba1 | — | — | — | — | — | — | — | — |
| | Ki67⁺ Nestin | ↓** | ↑** | ↑** | ↑* | ↓** | NS | NS | NS |
| | Ki67⁺ DCX | ↓** | ↑** | ↑** | ↑** | NS | NS | NS | NS |
| | Ki67⁺ PSA-NCAM | ↓** | ↑** | ↑** | ↑** | ↓** | NS | NS | NS |
| | Total NeuN | — | — | — | — | — | — | — | — |
| | Ki67⁻ NeuN | — | — | — | — | — | — | — | — |
| | Ki67⁺ NeuN | — | — | — | — | — | — | — | — |
| | Ki67⁺ A2B5 | NS | NS | NS | NS | ↓* | NS | NS | NS |
| | Total GFAP | — | — | — | — | — | — | — | — |
| | Ki67⁻ GFAP | — | — | — | — | — | — | — | — |
| | Ki67⁺ GFAP | — | — | — | — | — | — | — | — |
| 1 month | Total CD68 | NS | NS | NS | NS | NS | NS | NS | NS |
| | Ki67⁻ CD68 | NS | NS | NS | NS | NS | NS | NS | NS |
| | Ki67⁺ CD68 | NS | NS | NS | NS | NS | ↑* | NS | NS |
| | Total Iba1 | NS | NS | NS | ↓* | NS | NS | NS | NS |
| | Ki67⁻ Iba1 | NS | NS | NS | NS | NS | NS | NS | NS |
| | Ki67⁺ Iba1 | NS | NS | NS | NS | NS | NS | NS | NS |
| | Ki67⁺ Nestin | ↓* | NS | ↑* | NS | ↓* | NS | ↑* | NS |
| | Ki67⁺ DCX | NS | NS | NS | NS | NS | NS | NS | NS |
| | Ki67⁺ PSA-NCAM | NS | NS | NS | NS | ↓** | NS | ↑** | NS |
| | Total NeuN | NS | NS | NS | ↑** | NS | NS | NS | NS |
| | Ki67⁻ NeuN | NS | NS | NS | ↑** | NS | NS | NS | NS |
| | Ki67⁺ NeuN | NS | NS | NS | NS | NS | NS | NS | NS |
| | Ki67⁺ A2B5 | NS | NS | NS | NS | NS | NS | NS | NS |
| | Total GFAP | ↑** | NS | ↓* | NS | NS | NS | NS | NS |
| | Ki67⁻ GFAP | ↑** | NS | NS | NS | ↑** | ↓* | NS | ↓* |
| | Ki67⁺ FGAP | NS | NS | NS | NS | ↑** | ↑** | NS | NS |

HIE, HIE + Saline; Con, sham control; UCB, HIE + umbilical cord blood; MNC, HIE + mononucleated cells; RCF, HIE + red cell fraction; CD68 and Iba1 as repair markers; Nestin, DCX, PSA-NCAM and NeuN as neurogenesis markers; A2B5 and GFAP as gliogenesis markers; Ki67⁺, double Ki67 and other marker stained; Ki67⁻, only marker stained without Ki67; —, not available, NS, no significant; ↑, higher compared with Con or HIE; ↓, lower compared with Con or HIE; *, p ~ 0.05, **, p < 0.05.

HIE control groups have a decrease in neuogenesis in MC and SVZ at both 7 days and 1 month and a decrease in gliogenesis in SVZ in 7 days but an increase gliogenesis in MC and SVZ in 1 month, resulting in impaired locomotion and memory in both 7 days and 1 month.

MNC has the most therapeutic effects with an increase in neurogenesis in MC more than in SVZ in 7 days, an increase in neurogenesis in SVZ more than MC in 1 month, and a decrease gliogenesis in MC more than in SVZ in 1 month, resulting in improved both locomotion and memory in 7 days and 1 month.

UCB has less therapeutic effects when compared to MNC treatment with an increase in neurogenesis in MC more than in SVZ in 7 days, a decrease in gliogenesis in SVZ more than in MC in 1 month, and an increase macrophages in 1 month, resulting in improved locomotion in 7 days and 1 month and improved memory in 7 days only, not in 1 month.

RCF has minimal therapeutic effects with an increase in neurogenesis in MC more than in SVZ in 7 days, an increase in neurogenesis in SVZ more than in MC in 1 month, a decrease in microglia in MC in 1 month, and a decrease in gliogenesis in SVZ more than in MC in 1 month, resulting in no improved locomotion and memory in both 7 days and 1 month.

### EXEMPLARY EMBODIMENTS

Embodiment 1. A method of treating a neurological dysfunction in a subject, the method comprising administering umbilical cord blood (UCB), mononuclear cells (MNC), or red cell fractions (RCF) to the subject, whereby treatment of a neurological dysfunction results.

Embodiment 2. The method of Embodiment 1, wherein the neurological dysfunction is hypoxic-ischemic encephalopathy (HIE) or a stroke.

Embodiment 3. The method of Embodiment 1, wherein the UCB, MNC, or RCF are administered in a route selected from intravascular, intra-arterial, epidural, intracerebral, intradural, intrameningeal, intraspinal, intrathecal, subarachnoid, intranasal, and intra-tissue.

Embodiment 4. The method of Embodiment 3, wherein the administered UCB, MNC, or RCF remain in the circulatory system or in the tissue in which the UCB, MNC, or RCF are administered.

Embodiment 5. The method of Embodiment 3, wherein the UCB, MNC, or RCF are administered in epithelial tissue, connective tissue, brain tissue, or muscular tissue.

Embodiment 6. The method of Embodiment 1, wherein the administration of UCB, MNC, or RCF stimulates neurogenesis and incorporation of neurons and/or new neurons into neuronal circuitry of brain and spinal cord.

Embodiment 7. The method of Embodiment 1, wherein the administration of UCB, MNC, or RCF stimulates gliogenesis and incorporation of astrocytes and/or new astrocytes to repair a blood brain barrier of the brain and spinal cord.

Embodiment 8. The method of Embodiment 1, wherein the administration of UCB, MNC, or RCF stimulates vasculogenesis and incorporation of vascular cells and/or new vascular cells to repair vascular damage to at least one organ or tissue.

Embodiment 9. The method of Embodiment 8, wherein the organ or tissues is brain, spinal cord, bone, bone marrow, heart, lung, liver, pancreas, spleen, stomach, intestines, kidneys, muscles, guts, skin, hair, retina, hair organ, or any combination thereof.

Embodiment 10. The method of Embodiment 1, wherein UCB or MNC comprises exosomes and enriched lymphocytes, monocytes, progenitor cells, or any combination thereof with cell surface antigens CD45, CD34, CD133, CD14 or any combination thereof, and RCF comprises red blood cells, exosomes, and neutrophils.

Embodiment 11. The method of Embodiment 1, wherein the neurological dysfunction is treated within about 10 minutes to about 1 year after administration of UCB, MNC, or RCF.

Embodiment 12. The method of Embodiment 1, wherein the administration of UCB, MNC, or RCF enhances locomotor function, motor coordination, sensory perception, cognition, visual and auditory function, smell and taste function, memory, or any combination thereof.

Embodiment 13. The method of Embodiment 1, wherein the administration of UCB, MNC, or RCF replaces neurons and restores function in the motor cortex, thalamus, hypothalamus, hippocampus, Sensory cortex, auditory cortex, basil ganglia, cerebellum, olfactory bulb, retina, auditory hair cells, sensory receptors, or any combination thereof.

Embodiment 14. The method of Embodiment 6, wherein the neurons express Ki67 and Nestin, Ki67 and DCX, or Ki67 and PSA-NCAM.

Embodiment 15. The method of Embodiment 14, wherein the expression Ki67 and Nestin, Ki67 and DCX, or Ki67 and PSA-NCAM occurs in less 1 week after administration of UCB, MNC, or RCF to the subject.

Embodiment 16. The method of Embodiment 6, wherein the neurons express Ki67 and NeuN.

Embodiment 17. The method of Embodiment 16, wherein the Ki67 and NeuN occurs about 1 month to about 3 months after administration of MNC to the subject.

Embodiment 18. The method of Embodiment 1, wherein the quantity of cells from UCB, MNC, or RCF administered to the subject is about 1×10³ to about 1×10⁹.

Embodiment 19. The method of Embodiment 1, wherein the administered cells of UCB, MNC, or RCF do not cross a blood-brain barrier of the subject.

Embodiment 20. The method of Embodiment 1, wherein a limited amount of the administered cells of UCB, MNC, or RCF cross a blood-brain barrier of the subject.

Embodiment 21. The method of Embodiment 20, wherein less than about 50 cells per 25 mg of brain tissue cross the blood-brain barrier of the subject.

Embodiment 22. The method of Embodiment 1, wherein the UCB, MNC, or RCF comprise cytokines, growth factors, exosomes, cell free nucleic acids, proteins, or any combination thereof.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Palmer TD, Markakis EA, Willhoite AR, Safar F, Gage FH. Fibroblast growth factor-2 activates a latent neurogenic program in neural stem cells from diverse regions of the adult CNS. J Neurosci. 1999; 19(19):8487-97. Epub 1999/09/24. PubMed PMID: 10493749; PMCID: PMC6783019.
2. Gage FH. Adult neurogenesis in mammals. Science. 2019;364(6443):827-8. Epub 2019/05/31. doi: 10.1126/science.aav6885. PubMed PMID: 31147506.
3. Otani T, Marchetto MC, Gage FH, Simons BD, Livesey FJ. 2D and 3D Stem Cell Models of Primate Cortical Development Identify Species-Specific Differences in Progenitor Behavior Contributing to Brain Size. Cell Stem Cell. 2016;18(4):467-80. Epub 2016/04/07. doi: 10.1016/j.stem.2016.03.003. PubMed PMID: 27049876; PMCID: PMC4826446.
4. Toda T, Gage FH. Review: adult neurogenesis contributes to hippocampal plasticity. Cell Tissue Res. 2018;373(3):693-709. Epub 2017/12/01. doi: 10.1007/s00441-017-2735-4. PubMed PMID: 29185071.
5. Bhardwaj RD, Curtis MA, Spalding KL, Buchholz BA, Fink D, Bjork-Eriksson T, Nordborg C, Gage FH, Druid H, Eriksson PS, Frisen J. Neocortical neurogenesis in humans is restricted to development. Proc Natl Acad Sci USA. 2006; 103(33):12564-8. Epub 2006/08/2012. doi: 10.1073/pnas.0605177103. PubMed PMID: 16901981; PMCID: PMC1567918.
6. Jia Y, Xu H, Li Y, Wei C, Guo R, Wang F, Wu Y, Liu J, Jia J, Yan J, Qi X, Li Y, Gao X. A Modified Ficoll-Paque Gradient Method for Isolating Mononuclear Cells from the Peripheral and Umbilical Cord Blood of Humans for Biobanks and Clinical Laboratories. Biopreserv Biobank. 2018;16(2):82-91. Epub 20171212. doi: 10.1089/bio.2017.0082. PubMed PMID: 29232525.
7. Brun JF, Boulot P, Varlet-Marie E. Actual vs optimal fetal hematocrit measured with punctures of cord blood in utero: Relationship with umbilical artery resistance. Clin Hemorheol Microcirc. 2016;64(4): 789-97. doi: 10.3233/CH-168016. PubMed PMID: 27767969.
8. Christensen RD, Baer VL, Gerday E, Sheffield MJ, Richards DS, Shepherd JG, Snow GL, Bennett ST, Frank EL, Oh W. Whole-blood viscosity in the neonate: effects of gestational age, hematocrit, mean corpuscular volume and umbilical cord milking. J Perinatol. 2014;34(1):16-21. Epub 20130912. doi: 10.1038/jp.2013.112. PubMed PMID: 24030677.
9. Cervantes FJ, Giasi del Real R, Rodriguez JA, Jimenez Sanchez A. [Polycythemia in newborn infants. II. Umbilical cord and capillary hematocrits]. Ginecol Obstet Mex. 1989;57:8-15. PubMed PMID: 2486859.
10. Bernstine JB, Ludmir A. Hemoglobin and hematocrit studies in the newborn with ligated and nonligated umbilical cords. Am J Obstet Gynecol. 1959;78(1):66-8. doi: 10.1016/0002-9378(59)90642-8. PubMed PMID: 13661247.
11. Zhao YW, Man GCW, Chan LKY, Guo X, Liu YY, Zhang T, Kwong J, Wang CC, Chen XY, Li TC. 2021 Multiplexed fluorescent immunohistochemical staining of four endometrial immune cell types in recurrent miscarriage. JOVE-J Vis Exp 174;e62931:1-18. doi: 10.3791/62931 (video: see worldwide website: jove.com/v/62931).
12. Hao L, Sun DM, Ng Cp, Cheng W, Chen JF, He YZ, Lan SY, Zheng ZY, Huang GZ, Wang CC, Young W, Poon WS. Umbilical cord blood mononuclear cell treatment for neonatal rats with hypoxic ischemic.
13. Scholzen T, Gerdes J. The Ki-67 protein: from the known and the unknown. J Cell Physiol. 2000 Mar;182(3):311-22. doi: 10.1002/(SICI)1097-4652(200003)182:3<311::AID-JCP1>3.0.CO;2-9. PMID: 10653597.
14. Ayanlaja AA, Xiong Y, Gao Y, Ji G, Tang C, Abdikani Abdullah Z, Gao D. Distinct Features of Doublecortin as a Marker of Neuronal Migration and Its Implications in Cancer Cell Mobility. Front Mol Neurosci. 2017 Jun. 28;10:199. doi: 10.3389/fnmol.2017.00199. PMID: 28701917; PMCID: PMC5487455.
15. Lyu, H., Sun, D. M., Ng, C. P., Chen, J. F., He, Y. Z., Lam, S. Y., et al. (2021). A New Hypoxic Ischemic Encephalopathy Model in Neonatal Rats. Heliyon 7:e08646. doi: 10.1016/J.HELIYON.2021.E08646.
16. Shankaran, S., Laptook, A. R., Ehrenkranz, R. A., Tyson, J. E., McDonald, S. A., Donovan, E. F., et al. (2005). Whole-Body Hypothermia for Neonates with Hypoxic-Ischemic Encephalopathy. N. Engl. J. Med. 353, 1574-1584. doi: 10.1056/nejmcps050929

1. A method of treating a neurological dysfunction in a subject, the method comprising administering an effective amount of umbilical cord blood (UCB), mononuclear cells (MNC), or red cell fractions (RCF) to the subject, whereby treatment of a neurological dysfunction results.

2. The method of claim 1, wherein the neurological dysfunction is hypoxic-ischemic encephalopathy (HIE) or a stroke.

3. The method of claim 1, wherein the UCB, MNC, or RCF are administered in a route selected from intravascular, intra-arterial, epidural, intracerebral, intradural, intrameningeal, intraspinal, intrathecal, subarachnoid, intranasal, and intra-tissue.

4. (canceled)

5. The method of claim 3, wherein the UCB, MNC, or RCF are administered in epithelial tissue, connective tissue, brain tissue, or muscular tissue.

6. The method of claim 1, wherein the administration of UCB, MNC, or RCF stimulates neurogenesis and incorporation of neurons and/or new neurons into neuronal circuitry of brain and spinal cord.

7. The method of claim 1, wherein the administration of UCB, MNC, or RCF stimulates gliogenesis and incorporation of astrocytes and/or new astrocytes to repair a blood brain barrier of the brain and spinal cord.

8. The method of claim 1, wherein the administration of UCB, MNC, or RCF stimulates vasculogenesis and incorporation of vascular cells and/or new vascular cells to repair vascular damage to at least one organ or tissue.

9. The method of claim 8, wherein the organ or tissues is brain, spinal cord, bone, bone marrow, heart, lung, liver, pancreas, spleen, stomach, intestines, kidneys, muscles, guts, skin, hair, retina, hair organ, or any combination thereof.

10. The method of claim 1, wherein UCB or MNC comprises exosomes and enriched lymphocytes, monocytes, progenitor cells, or any combination thereof with cell surface antigens CD45, CD34, CD133, CD14 or any combination thereof, and RCF comprises red blood cells, exosomes, and neutrophils.

11. The method of claim 1, wherein the neurological dysfunction is treated within about 10 minutes to about 1 year after administration of UCB, MNC, or RCF.

12. The method of claim 1, wherein the administration of UCB, MNC, or RCF enhances locomotor function, motor coordination, sensory perception, cognition, visual and auditory function, smell and taste function, memory, or any combination thereof.

13. The method of claim 1, wherein the administration of UCB, MNC, or RCF replaces neurons and restores function in the motor cortex, thalamus, hypothalamus, hippocampus, Sensory cortex, auditory cortex, basil ganglia, cerebellum, olfactory bulb, retina, auditory hair cells, sensory receptors, or any combination thereof.

14. The method of claim 6, wherein the neurons express Ki67 and Nestin, Ki67 and DCX, or Ki67 and PSA-NCAM.

15. The method of claim 14, wherein the expression Ki67 and Nestin, Ki67 and DCX, or Ki67 and PSA-NCAM occurs in less 1 week after administration of UCB, MNC, or RCF to the subject.

16. The method of claim 6, wherein the neurons express Ki67 and NeuN.

17. The method of claim 16, wherein the Ki67 and NeuN occurs about 1 month to about 3 months after administration of MNC to the subject.

18. The method of claim 1, wherein the quantity of cells from UCB, MNC, or RCF administered to the subject is about 1×10³ to about 1×10⁹.

19. The method of claim 1, wherein the administered cells of UCB, MNC, or RCF do not cross a blood-brain barrier of the subject.

20. The method of claim 1, wherein a limited amount of the administered cells of UCB, MNC, or RCF cross a blood-brain barrier of the subject.

21. The method of claim 20, wherein less than about 50 cells per 25 mg of brain tissue cross the blood-brain barrier of the subject.

22. (canceled)
